(19) United States
(12) Patent Application Publication
(10) Pub. No.: US 2008/0139395 A1
(43) Pub. Date: Jun. 12, 2008

(54) Microarray and Spotting Apparatus

(76) Inventors: Ryoichi Imanaka, Osaka (JP); Kotaro Minato, Kyoto (JP); Tadao Sugiura, Osaka (JP)

Correspondence Address: KUBOVCIK & KUBOVCIK, SUITE 1105, 1215 SOUTH CLARK STREET, ARLINGTON, VA, 22202, US

(21) Appl. No.: 10/591,147

(22) PCT Filed: Mar. 4, 2005
(86) PCT No.: PCT/JP05/04263

§ 371 Date: Aug. 30, 2006

(30) Foreign Application Priority Data
Mar. 4, 2004 (JP) 2004-61307

(51) Int. Cl. C40B 20/02; C40B 50/14; C40B 60/14; C07H 21/00

(52) U.S. Cl. 506/3; 506/30; 536/25.31; 506/40

(57) ABSTRACT

A microarray disc characterized in that a substrate is provided with a pregroove and a thin film with an excellent adherence to a probe DNA or protein is disposed at least on the pregroove, and that a liquid drop containing the probe DNA or protein is arranged on a convex part or concave part of the pregroove so that the liquid drop expands in the tangential direction of the pregroove due to the surface tension of the liquid drop and/or in the instance of concave part, due to the restriction by the concave groove wall with any expansion in the direction perpendicular to the groove, and that in the above condition, the probe DNA or protein is immobilized on the substrate.

### TECHNICAL FIELD

This invention relates to constitution of a microarray and microarray disc and spotting apparatus therefore.

### BACKGROUND ART

A DNA microarray is the one which is fixed with thousands of probe DNA on a substrate, such as slide glass, passes a sample (target) DNA which is labeled by fluorescence molecule etc., is made to hybridize with the probe DNA, measures the strength of detection of fluorescence luminescence caused by the hybrid formation, and estimates the amount of the gene expression contained in the sample.

Because DNA microarray can analyze a lot of gene expressions comprehensively and simultaneously, it has spread through the research and development field in life science, pharmacy, and agriculture as a basic standard apparatus. For example, for the two samples of mRNA (messenger RNA), one is extracted from a cancer tissue which does not react with a certain medicine and the other is extracted from another cancer tissue which does react with the certain medicine. The difference of both amounts of gene expressions, which is measured by DNA microarray in which all probes of human genome are included, will make clear the specific genes expressions related to the cancer tissue and the medicine. Using the gene as a research target, elucidating the disease dynamics or new drug research can be done efficiently.

It goes into the time of a post genome sequence, and the necessity for comprehensive analysis of gene expressions spreads to new biotechnology industries, such as food inspection and a production quality control, as well as the laboratory test in so-called the tailor made medicine and the clinical diagnosis. A big DNA microarray market is expected to grow from now on.

There are two major types of DNA microarray. One is a combination of lithographic techniques and DNA producing, and is called Affimetrix type, which accumulated oligonucleotide perpendicular on silicone substrate, and the other is called Stanford type, which spots DNA on slide glass. Although the former must choose a gene beforehand, must request order, design, and manufacture beforehand and is expensive, the latter has an advantage that a user can freely choose the gene in his own laboratory environment. In this invention, a probe DNA (after hybridization, this is called DNA spots) means the form of the Affimetrix type, a Stanford type described above, and other types of a probe DNA.

A conventional DNA microarray is shown in FIG. 11. DNA spots sequence 111 arranged in the format of a two-dimensional lattice on glass substrate 110 is read by detecting and picturizing fluorescence thereof by means of a laser scanning or an image measurement. As two dimensions need to be measured, the equipment becomes complicated and moreover compensation of detecting fluorescence signal and computational picture processing for noise reduction are required as post processing. Although there are other kinds of equipment using a bead or a porous medium instead of a slide glass, the number of probes simultaneously be treated is limited at most several hundreds, thus the comprehensibility is insufficient.

However, the system which consists of a spotter, a hybridization apparatus and a scanner for analyzing DNA microarray has many problems from the points of quantitative accuracy and sensitivity.

The problems to be solved are itemized below:

### 1. The sensitivity of detection of fluorescence measurement is low.

### 2. Reading takes lot of time.

### 3. Two dimensional scanning is required.

### 4. Operability is poor.

### 5. The number of DNA spots arranged on a glass substrate is not enough (at most 10,000 spots).

### 6. Because recording the characteristic of a sample and experimental conditions simultaneously is difficult, numbers are added to the sample and the extra data sheet is required separately.

The purpose of this invention is to improve fundamental elements of DNA microarray, which are the structure of spotting apparatus, a probe DNA production apparatus or photochemical reaction generates, and to realize a new system which can read robustly, obtain the analysis result rapidly with a simple composition and to offer a reasonable price with a high efficiency.

The spots of DNA microarray, which are conventionally arranged in the shape of two-dimensional lattice in the X and Y coordinates, are made to arrange on one dimensional line, simultaneously the glass substrate is changed into a disc form, and indices are formed, such as pregroove and prepit to identify a spot position. Thus, it is an invention to offer the DNA microarray disc, which is created by spotting a probe DNA on the pregroove, and the apparatus for spotting. This invention relates to a method of generating a probe DNA by spotting or photochemical reaction especially to substrate of DNA microarray used for Japanese Patent Publication (laid open) No. 2004-333333 by the same inventor, and to substrate excellent in detection of fluorescence (fluorescence detection) measurement sensitivity.

### DISCLOSURE OF THE INVENTION

This invention relates to the following inventions.

A. A microarray disc characterized in that a substrate is provided with a pregroove and a thin film with an excellent adherence to probe DNA or protein is disposed at least on the pregroove, and that a liquid drop containing a probe DNA or protein is arranged on a convex part or concave part of the pregroove so that the liquid drop expands in the tangential direction of the pregroove due to the surface tension of the liquid drop and/or in the instance of concave part, which is restricted with any expansion in the direction perpendicular to the groove by concave groove wall, and that in the above condition, the probe DNA or protein is immobilized on the substrate.

B. A spotting apparatus to produce a microarray disc in order to arrange liquid drops of probe DNA or protein on a convex part or concave part of the pregroove, which comprises spotting liquid drops containing probe DNA or protein on the pregroove by 1) means of detecting the position of the pregroove, and 2) means of discharging liquid drops containing probe DNA or protein.

C. A microarray disc characterized in that at least one layer including a thin film on the substrate is provided, when detecting a position of pregroove on the disc, a laser beam of wavelength λ1 is irradiated from the substrate side and the laser beam which irradiates the above-mentioned substrate, is partially penetrated; and when measuring a spot of DNA or protein arranged on the substrate, a laser beam with a detection wavelength λ2 irradiated from the opposite side of the substrate is partially reflected.

D. A spotting apparatus to produce a microarray disc comprising 1) means of detecting the position of the pregroove, and 2) means of discharging liquid drops containing probe DNA or protein in order to arrange liquid drops of probe DNA or protein on a convex part or concave part of the pregroove on the disc, which further includes an optical measurement mechanism in order to control the discharging position of a liquid drop and the amount of the liquid drop, and includes a controller and a mechanism in order to detect a pregroove on the disc and to form liquid spots on the pregroove.

E. A spotting equipment that has a mechanism, an optical measurement part and a controller (it is also called servo mechanism) in order to spot different kinds of a probe DNA or a protein on a substrate efficiently, detecting a relative position between a pregroove and a nozzle such as a inkjet which discharges a liquid drop containing a probe DNA or a protein, a micro pipette which is a discharge device dropping a liquid spot, or a needlelike tool, controlling a nozzle unit movement which has multiple nozzles, and arranging different kinds of a probe DNA or a protein one after another in the predetermined position of the pregroove.

F. A method of producing a probe DNA which irradiates a laser beam selectively with detecting the address that represents the location of the pregroove, in order to generate a probe DNA by photochemical reaction in a convex part or concave part of the pregroove on DNA microarray disc.

G. A probe DNA generating apparatus characterized in that a substrate is provided with an identified addressable flat location by a pregroove or prepit and thin film is provided on the substrate with an excellent adherence to probe DNA at least on the pregroove or the flat location, and when an oligonucleotide is generated by photochemical reaction in a convex part and/or concave part of the pregroove or storing location of probe DNA, the storing location is identified by the address information of the pregroove or prepit, after irradiating and activating a laser light at the location, the first monomer is applied, then irradiating a laser light to one photoremovable protective group of the above monomer and the photoremovable protective group is removed, and the 2nd monomer is applied and bound, so that generating any oligonucleotide.

H. Microarray disc characterized in that the quality is examined about plural spots of same kind of probe DNA or protein and the address information of the location of the probe DNA or protein, which is judged at least as a proper quality, is kept.

### BRIEF DESCRIPTION OF THE DRAWINGS

FIG. 1 shows an outline constitution of the spotting apparatus, which is one embodiment of this invention.

FIG. 2 shows an outline constitution of DNA microarray disc of this invention.

FIG. 2a is an enlargement view of the address information and V MARK of DNA microarray disc of this invention.

FIG. 3 is a block diagram showing a controller of a spotting apparatus in one embodiment of this invention.

FIG. 4 shows a reflective quantity of light distribution change at spotting.

FIG. 5 shows an outline of a spotting apparatus, which has two or more nozzles for discharging a spotting liquid, i.e. probe DNA.

FIG. 6 is a side view of an outline of multi-spotting apparatus.

FIG. 7 is a top view of an outline of multi-spotting apparatus.

FIG. 8 is a block diagram showing a controller of multi-spotting apparatus.

FIG. 9 shows an outline of an inkjet and a spotting liquid tank.

FIG. 10 is a graph which shows calculation results of electric field intensity for the gold or the SiO₂ thin film formed on gold thin film on DNA microarray disc.

FIG. 11 shows a conventional DNA microarray constitution.

FIG. 12 is an enlargement view of a DNA microarray disc pregroove.

FIG. 13 is a block diagram of probes DNA generating apparatus.

FIG. 14 is a control block diagram of probe DNA generating apparatus.

FIG. 15 is a waveform chart of probe DNA generating apparatus.

FIG. 16 is a block diagram of a reading apparatus.

FIG. 17 is an arrangement view of a reading-beam and a servo-beam in DNA microarray disc.

### EXPLANATION OF SYMBOLS

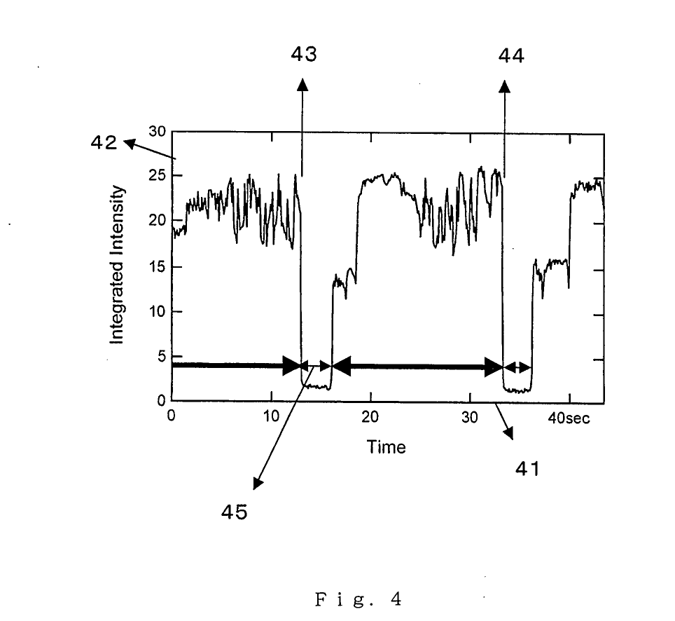

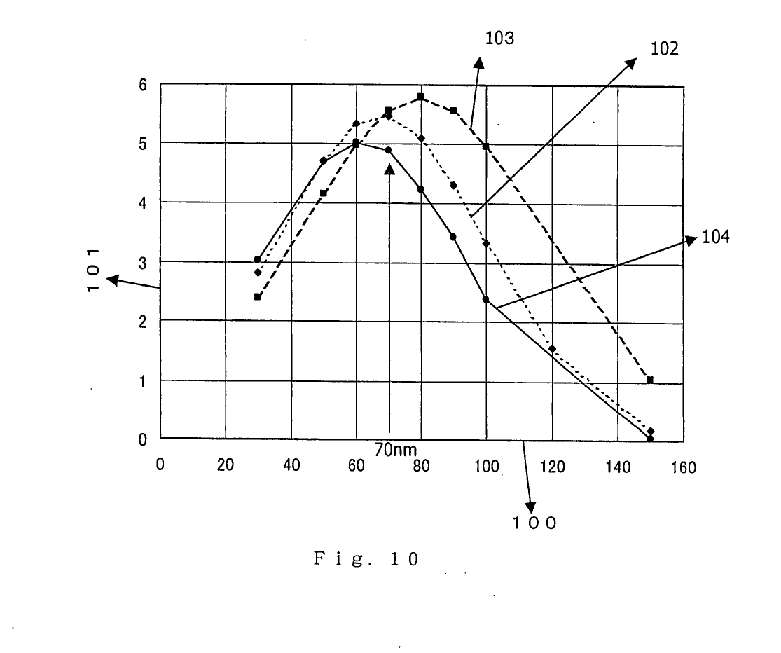

1 DNA microarray disc, 2 Laser, 3 Beam Expander, 4 Object lens, 4a Tracking actuator, 4b Focusing actuator, 5 Beam splitter, 6 Lens, 7 Photo detector, 7a Photo detector A, 7b Photo detector B, 8 Inkjet, 9 Inkjet nozzle, 10 Photo detector, 10a Photo detector A, 10b Photo detector B, 11 Spotting liquid supply tube, 12 Traverse unit A, 12-1 traverse unit B, 12-2 traverse unit C, 13 Traverse motor A, 13-1 Traverse motor B, 13-2 Traverse motor C, 14 Disc motor, 15 V MARK Detector, 22 Center hole, 23 Pregroove, 24 first data recording region, 25 2nd data recording region, 26 V MARK, 26a V MARK pit “a” sequence, 26b V MARK pit “b” sequence, 27 Address information of pregroove, 28 Radial direction, 29 Tangential direction, 30 Differential amplifier 1, 31 Phase compensator amplifier 1, 32 Drive amplifier 1, 33 Differential amplifier 2, 34 Phase compensator amplifier 2, Drive amplifier 2, 36 CPU, 37 Adder amplifier, 38 Adder amplifier output, 39 Disc motor controller, 41 Horizontal axis of FIG. 4; DNA microarray disc rotation lapse time, 42 Reflection quantity of light (vertical axis in FIG. 4), 43 The spotting time, 44 The spotting time, 45 A reflective quantity of light fall period, 51 Inkjet, 52 Inkjet nozzle, 53 Inkjet unit, 54 Inkjet unit moving direction, 55 Transfer gear, 61 Disc motor transfer equipment, 62 Transfer direction, 71 Multi-inkjet rotary table, 81 Rotary table rotation controller, 82 Phase compensator amplifier 3, 83 Drive amplifier 3, 90 Inkjet A, 91 A tank, 92 A connection seal, 93 Nozzle(s). connection hole, 94 emitting hole, 95 A pressurization room, 96 A pressurization device, 97 A liquid name display part, 100 Horizontal axis of FIG. 10: thickness of SiO₂ film [nm], 101 Vertical axis of FIG. 10: electric field intensity on the substrate surface, 102 the characteristic with a wavelength of 563 nm, 103 The characteristic with a wavelength of 652 nm, 104 The characteristic with a wavelength of 532 nm, 110 Glass substrate, 111 DNA spots sequence, 112 Pregroove of addresses 1, 113 Address 1, 114 Pregroove of address 2, 115 Address 2, 116 Radial direction, 117 Tangential direction, 118 DNA microarray disc, 120 Laser, 121 Beam expander, 122 Beam splitter, 123 Object lens, 124 Tracking actuator, 125 Focusing actuator, 126 Lens, 128 Photo detector, 129 Traverse unit, 130 Traverse motor, 131 Disc motor, 132 table, 140 Preamplifier, 141 Decoder, 142 CPU, 143 Irradiate pulse controller, 144 I/F (Interface), 145 PC (Personal Computer), 146 Laser power modulator, 147 servo part, 148 Reproducing power, 149 Peak power, 152 Disc motor, 153 Object lens, 153a X direction, 153b Z direction, 154 Object lens actuator, 154a Tracking element, 154b Focusing element, 155 Excitation light source for detection of fluorescence (output light wave length λ1), 156 Collimator lens 1, 157 Light source for servo (output light wave length λ3), 158 Collimator lens 2, 159 Half mirror, 160 Beam splitter 1, 161 Condenser lens 1, 162 Optical filter, 163 Fluorescence light detector, 164 Beam splitter 2, 165 Condenser lens 2, 166 Servo error photo detector, 167 A Cylindrical lens, 168 A Diffraction lattice, 170 DNA spots reading beam, 171 Pregroove or a DNA spots sequence, 172 The beam for servo, 173 DNA spots, 174×direction.

### BEST MODE OF CARRYING OUT THE INVENTION

In preferred embodiments of the present invention, DNA microarray has one-dimensional arrangement such as concentric circles or spiral shape; contrary to the conventional DNA microarray having a two-dimensional arrangement of spots, has a disc shape of glass plate, and has an index, which can specify a spot position. In the concrete, spotting can be precisely applied to pregroove on a glass disc or indexed positions on the disc. And DNA spot can be read without distortion by scanning in one-dimensional direction. This invention concerns to a DNA microarray disc and an apparatus to spot probe DNA on the DNA microarray disc.

Furthermore, a recordable zone is formed on a DNA microarray disc, and the corresponding information on preparation conditions, measured results, the address information of spotting points indexed by pregroove and the name of the corresponding spotting liquid are recorded on the DNA microarray disc. Accordingly, a glass plate and preparation conditions, which are conventionally stored separately, can be stored simultaneously on the microarray disc. As a result, operability, reliability and security of the DNA microarray disc are improved. The present invention provides concrete production methods for the purpose.

The followings are the best mode for carrying out this invention indicated together with drawings.

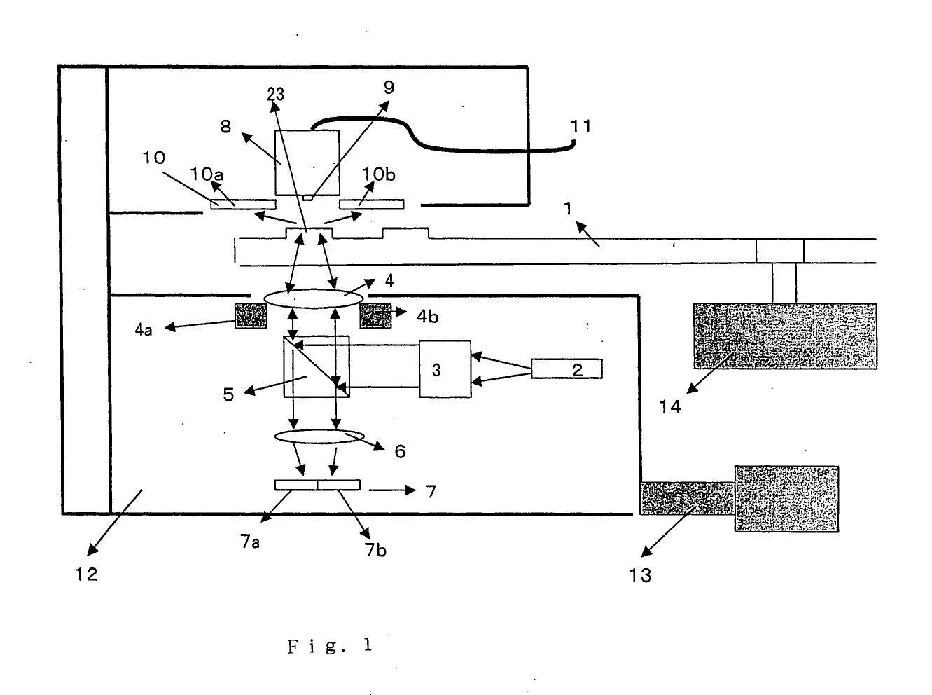

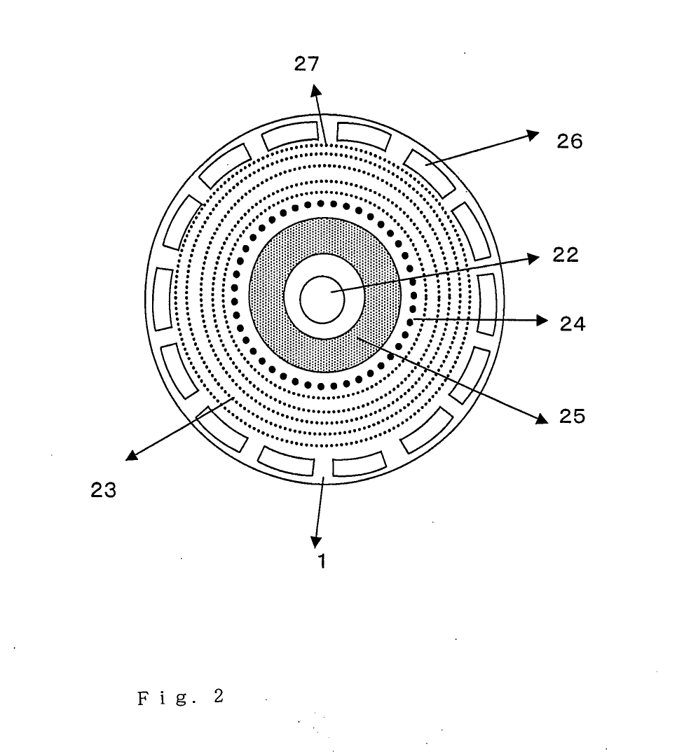

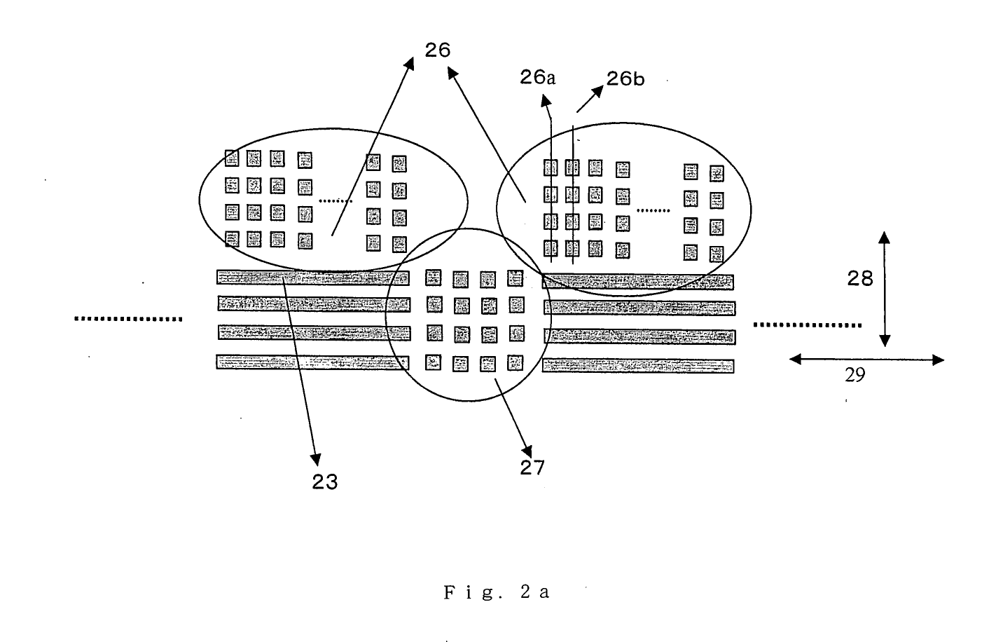

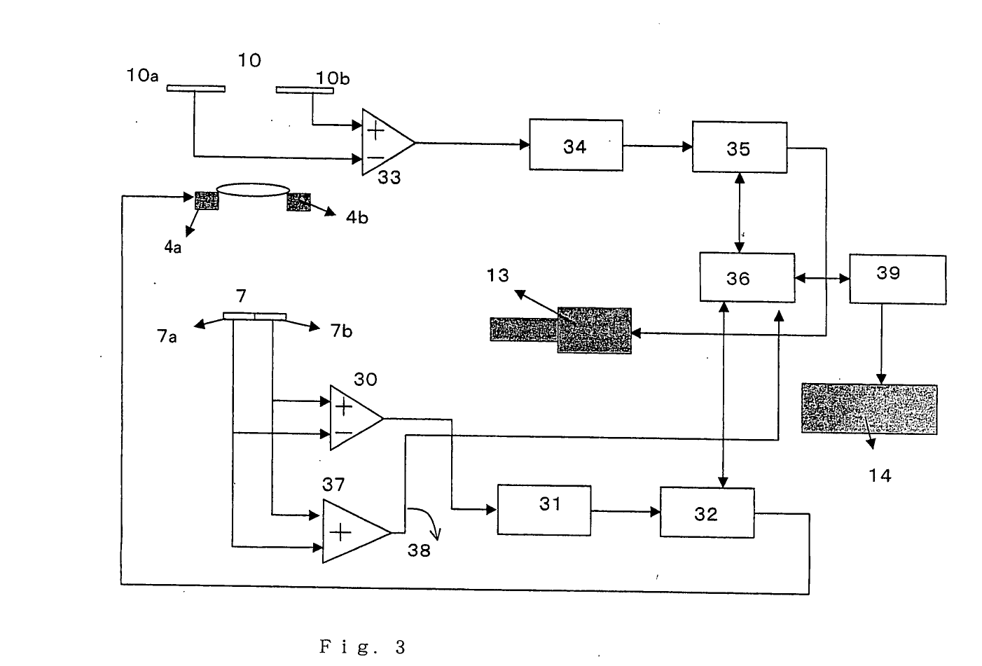

Hereafter, the outline constitution of the first case of this invention is illustrated in FIG. 1, FIG. 2, and FIG. 3. In addition, although protein etc. is also possible to be used, DNA is mainly used for explanation hereafter.

FIG. 1 shows a block diagram of an apparatus for spotting probe DNA on a DNA microarray disc, which is depicted in FIG. 2. FIG. 2 shows the DNA microarray disc.

In FIG. 1, 1 is the DNA microarray disc and has a pregroove 23. And a disc motor 14 controls the rotation. 8 is a discharge apparatus of probe DNA, which uses an inkjet nozzle for spotting probe DNA on the substrate. This apparatus can be formed also with a micro pipette or a tool like a nib. To detect the position of the above-mentioned inkjet, a photodetector 10 (it has detection elements 10a and 10b) is put at a fixed position relative to the nozzle of the inkjet. In addition, forming of DNA spots, which are spotted and are immobilized on pregroove, is experimentally confirmed that the size in longitudinal direction of a spot is greater than that in the perpendicular direction and the ratio of the former versus later is equal to or greater than two.

Spotting liquid of probe DNA is supplied from a tank formed in the inkjet or from a spotting-liquid supply tube 11. Spotting liquid is solution, that probe DNA and protein, etc. are dissolved in water or other media (alcohol etc.). This spotting liquid can be placed on both or either convex or concave part of the pregroove.

2 is a laser. A beam from the laser 2 is changed into parallel light by an expander 3 or a collimator lens, and is focused onto a pregroove 23 on the DNA microarray disc 1 with an object lens 4 through a beam splitter 5. Reflected light from the pregroove 23 travels through the objective lens 4 and the beam splitter 5 and forms a far field image of the pregroove on the photodetector 7, which are comprised of a set of two-segmented cell 7a and 7b. From a differential signal of the two-segmented cell 7a and 7b, it is possible to detect a relative position between the beam spot, outputted from the object lens 4, and the pregroove 23.

Moreover light penetrating through the pregroove 23 is incident to the photodetector 10, which are comprised of two detection cells 10a and 10b. And the differential signal of the detecting cells 10a and 10b shows a relative position between the inkjet nozzle 9 and the pregroove. 12 is a traverse unit A. That is comprised of a photodetection part and the mechanical part, which include the inkjet 8, the photodetector 10, laser 2, the beam expander 3, the object lens 4, actuators 4a and 4b, the beam splitter 5, the lens 6, photodetector 7, and is controlled in the radial direction of the DNA microarray disc 1 by the traverse motor A.

A controller drives the object lens 4 in radial direction of the DNA microarray disc so that the laser beam from the object lens follows the pregroove 23. The controller is called tracking servo. And a photodetector 10 detects the position of the inkjet 8, and the controller, which aligns the liquid discharged from the inkjet on the pregroove 23, is called traverse servo.

In FIG. 2, on the DNA microarray disc 1a main hole 22 for rotating by a disc motor and convex or concave pregroove 23 are formed. As a method of creating pregroove, selective etching on a glass substrate can form a pregroove. Moreover, printing can also form the convex part of pregroove. When printing forms the pregroove, DNA spot is aligned on the part of substrate where the ink for printing does not adhere. It is also possible to create pregroove by injection mold which is a same method as optical discs such as CD, using resin, of course. Moreover, a pregroove can be cut in the tangential direction, and it can be considered as prepits, and can be used as prepits instead of a pregroove.

On the surface where probe DNA is spotted, thin film such as SiO₂ or gold, which does not emit fluorescence by laser beam irradiation, are formed if necessary. Furthermore, on the thin film, such as SiO₂ or gold, a thin film having an excellent adhesiveness to probe DNA is formed. The latter thin film promotes adhesion with the probe DNA to be spotted. When a droplet of probe DNA solution is spotted at concave part, it stays on a pregroove by the surface tension with existence of the wall of concave part. And as the liquid that dissolves probe DNA evaporates with a lapse of time, finally probe DNA is fixed on a pregroove.

Although a representative example of such thin film is one formed by processing a microarray with poly L-lysine (poly-L-lysine: PLL as abbreviation) solution, it is not limited to this and 3-aminopropyltriethoxysilane (APS as abbreviation) solution etc. can also be used. DNA, which is biotinated at terminal, can be used as probe DNA, By fixing avidin to the substrate surface, DNA can be fixed to the substrate with the site-specific conjugation between avidin and biotin. Moreover, it is more desirable to form a thin film symmetrically with both sides of a substrate, since the substrate might bend by change of temperature-humidity condition when the thin film is once formed only one side of the surface of the substrate. It is desirable to form the thin film as the laser beam can penetrate the substrate from the substrate side.

The address information to identify the position of a pregroove is added to the pregroove. In FIG. 2, a pregroove is created in the shape of a concentric circle, or in the shape of spiral. As shown in 27, a part of pregroove of a concentric circle is cut, the portion with pregroove and the portion without pregroove are formed, and it is considered as the address information, which shows the position of the pregroove. Moreover, the mark which shows a rotation position called a V mark 26 is formed in the most outer part or inner part of a DNA microarray disc. FIG. 2a is an enlarged picture of the address information and a V mark shown in a part of FIG. 2. Since an actual pregroove goes along the circumference, it becomes fan-shape. However, it is shown as a straight line for simplification. In addition, the corresponding relationship with FIG. 2 is given and shown with the same number.

The DNA spot by below-mentioned spotting on the DNA microarray disc above-mentioned is also called a DNA microarray disc.

Next, the constitution of a V mark is described in detail. In FIG. 2a, this V mark 26 shows the angle of the rotation direction and the prepit sequence (26a, 26b--) is formed, which shows the address information on a pregroove to the angle of the rotation direction between the V marks 26 in the present working example. Thus, by preparing a V mark, it becomes easy to detect the position on the disc circumference of address information. For example, if 26a is made into zero starting point, 26b will become the index, which shows the position of 0.5 degree on the circumference.

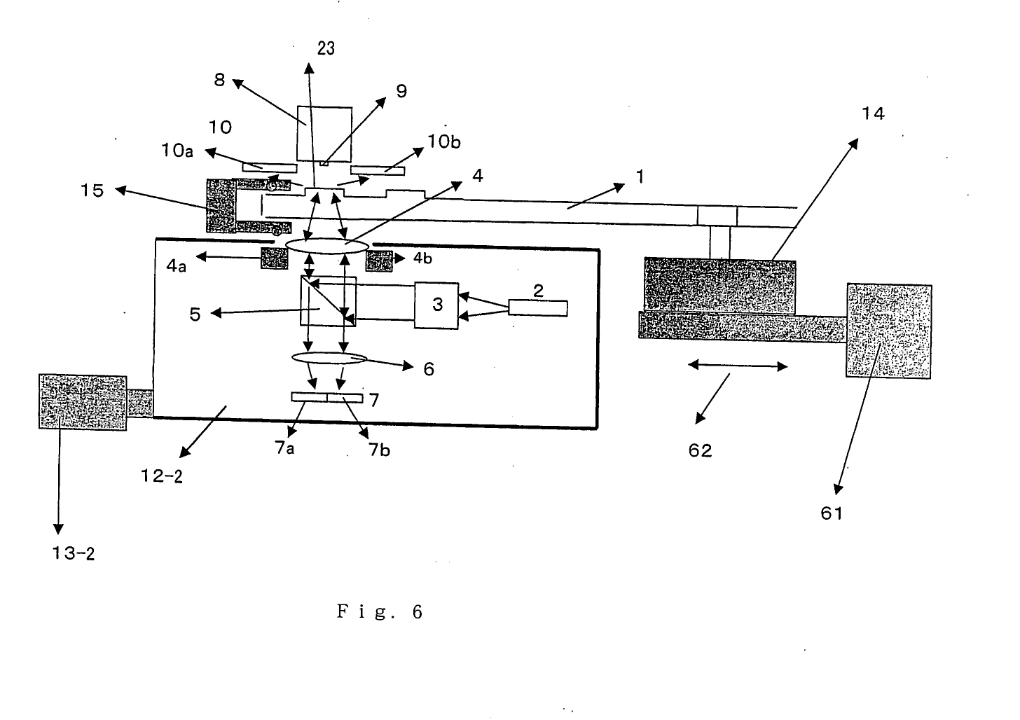

Thus, a V mark will constitute the absolute address showing a rotation angle on a disc. For example, the prepit sequence (26a, 26b--), which expresses 4 bits in the radius direction, is constituted, and the angle is made to correspond to an address using them. In addition, the arrow 28 in FIG. 2a shows the radius direction of a DNA microarray disc, and the arrow 29 shows the tangential direction. The address information on the pregroove 27 shows the address information, which indicates the position of the radius direction of the pregroove aligned two or more in the radius direction. (For example, the most outer circumference is made into the first pregroove and the 4-bit address used as 2, 3, and 4 is given in the direction of an inner circumference). While recording address information and angle information, well-known modulation methods, for example FM method and a phase modulation method, are used. The angle information, which a V mark shows, is read by a V mark detector 15 shown in FIG. 6, and the position information on the pregroove is read by the light beam from the object lens 4, which scans the pregroove. When a time lag on a time-axis exists, of course, it is modified by CPU 36 of spotting equipment.

Moreover, in FIG. 2, the first data recording region 24 is made of the ink etc., which can generate a contrast mark in order to use spotting liquid for data recording at the time of spotting or at the end of spotting. And this is for recording of information by changing the position, the size, and the phase, etc., of spots produced from this ink etc. And when DNA spot is set on the substrate, the sequence of the recording spots is modulated by information, such as conditions of forming the DNA spots on a substrate, and is recorded.

Especially it is effective to record the address information, which shows the spotting position, and corresponding relations between the spots and names of spotting liquid.

It is possible to use the following modulation methods: the positions of record spots are modulated with binary values of signals as expressing the information signal, or the periods of recording spots are modulated according to the information signal etc. Moreover, as a material, which constitutes record spot, the ink made from organic or inorganic material can be used.

Moreover, although it is needless to say, the corresponding relations between the address information and the name of spotting liquid on a DNA microarray can be recorded on another memory, and can also be attached to the DNA microarray. In this case, a barcode etc. can be used as identifying information of the DNA microarray, and the relation with the memory can be recorded on the memory.

After reading The DNA spots, the 2nd data recording region 25 on the substrate, which is independent from the first data recording region, also made it possible to record additionally the information data from read-out of The DNA spots.

For a recordable region, dye material can be used, which is conventionally used for an optical recordable disc, or a metal thin film can be formed by vacuum evaporation or spattering process on a substrate. It is desirable for the 2nd data-recording region to be formed on a pregroove, and to be addressed with the above-mentioned pregroove, and to discriminate easily from other regions. Of course, it is also possible for the data-recording region to record the address information of spotting, and the corresponding relation between spot and a name of spotting liquid. It is also possible to store the information acquired while spotting on another memory temporarily, and to record it collectively, after completing spotting.

Next, an operation is explained by using FIG. 2 with a DNA microarray disc, FIG. 3 with the block diagram of the controller of spotting apparatus and FIG. 4 with the graph, which shows the change of intensity distribution of reflected light while spotting on the DNA microarray disc.

In FIG. 1 and FIG. 3, the laser beam from the object lens 4 follows the pregroove 23 on the DNA microarray disc 1 by the tracking actuator 4a. For this reason, the output difference of the photodetectors 7a and 7b, which form the photodetector 7, is acquired as an output of the differential amplifier 30, compensates the response of a tracking servo part through the phase compensator amplifier 31, and controls the position of the tracking actuator 4a with the drive amplifier 32.

Moreover, a part of the beam from the object lens 4 penetrates the DNA microarray disc 1, and is detected by the photodetector 10, which includes photodetectors 10a and 10b, and then the difference is outputted by the differential amplifier 33. The output from the differential amplifier 33 gives an appropriate response of a traverse servo through the phase compensator amplifier 34, with the drive amplifier 1 (35), carries out the control of the traverse motor A 13, and the position of the traverse unit A 13 is controlled. Consequently, the position of inkjet nozzle 9 is controlled to follow the pregroove 23, and spotting liquid is discharged from the inkjet and is aligned on the pregroove 23.

Spotting liquid, which is discharged from the inkjet and is adhered on the pregroove, is irradiated by a beam spot from the object lens 4. And the light is reflected by the pregroove and adhered spotting liquid and is detected by the photodetector 7. FIG. 4 is a graph showing the result. The horizontal axis of the graph in FIG. 4 shows the rotational position of a DNA microarray disc, and the vertical axis shows the amount of the light detected with the photodetector 7.

FIG. 4 shows that the amount of the reflected light decreases remarkably by spotting liquid, when spotting liquid adheres on a pregroove. This is shown by the arrow 45 in FIG. 4, which indicates the position of intensity decrease of the reflected light.

The photodetector 7 can detect the timing when spotting liquid is discharged and is adhered on the pregroove. In this embodiment, the output of the photodetector 7 is detected by the adder amplifier 37 the adder output 38 is supplied to the CPU 36. It is possible to distinguish whether spotting liquid adhered or not and also possible to measure the adhered position by the output of the photodetector 7. The position of the traverse unit A 13 is controlled by measuring the output of the above-mentioned photodetector 7. Since the accuracy of discharge position using the inkjet is about 30 micrometers, spotting liquid can be controlled so that the liquid is aligned on pregroove, when the pitch of the radial direction on a pregroove (it is called a track pitch) is set to 30 micrometer. Moreover, regarding the position accuracy in the tangential direction of a pregroove, it can be adjusted to the optimal position by aligning the attachment position of an inkjet.

Next, the adjustment method of attachment position of the inkjet is concretely explained using FIG. 1 and FIG. 3. The laser light from the laser in FIG. 1 is focused on the pregroove 23 through the object lens. Then spotting liquid is discharged from the inkjet nozzle 9.

The operation at this time is depicted in FIG. 4. In FIG. 4, the DNA microarray disc 1 is made to rotate and spotting liquid is discharged at the point of discharging as shown 43 and 44. The intensity of the light, which reflects back from the pregroove 23 onto the photodetector 7 via the object lens, was plotted on the vertical axis 42 in the graph. The horizontal axis shows time 41 as past time on rotation of the DNA microarray disc.

When spotting liquid adheres on the pregroove, the intensity of the light, which reflects from the pregroove and returns to the photodetector 7 via the object lens, becomes about 1/10 as shown in the period 45 in FIG. 4. The distribution of the reflected light from the pregroove 23 is measured by the photodetector 7. Then the position of the inkjet 8 is adjusted so that spotting liquid discharged is aligned onto the center of pregroove 23. Adjustment is repeated by discharging from an inkjet several times, and then position is set to the optimal position.

A quantity of light distribution is measured by the differential amplifier 30 based on the output of the photodetector 7 and the intensity of light is measured by supplying the output from the adder 37 to CPU 36 so that the position of traverse unit A 13 is controlled.

In addition, the disc motor 14 drives the rotation of the DNA microarray disc 1.

In addition, the object lens 4 can be moved in the direction of perpendicular to a DNA microarray disc by object lens actuator 4b. By detecting the distance between the microarray disc 1 and the object lens 4, and by holding the distance in constant, the focal control part (not shown) can be obtained. At this time, the relative position between the object lens 4 and the DNA microarray disc 1 is detected, and drive control of the position (perpendicular to a DNA microarray disc) of the object lens 4 is carried out by the focusing actuator 4b so that the detected position is kept constant.

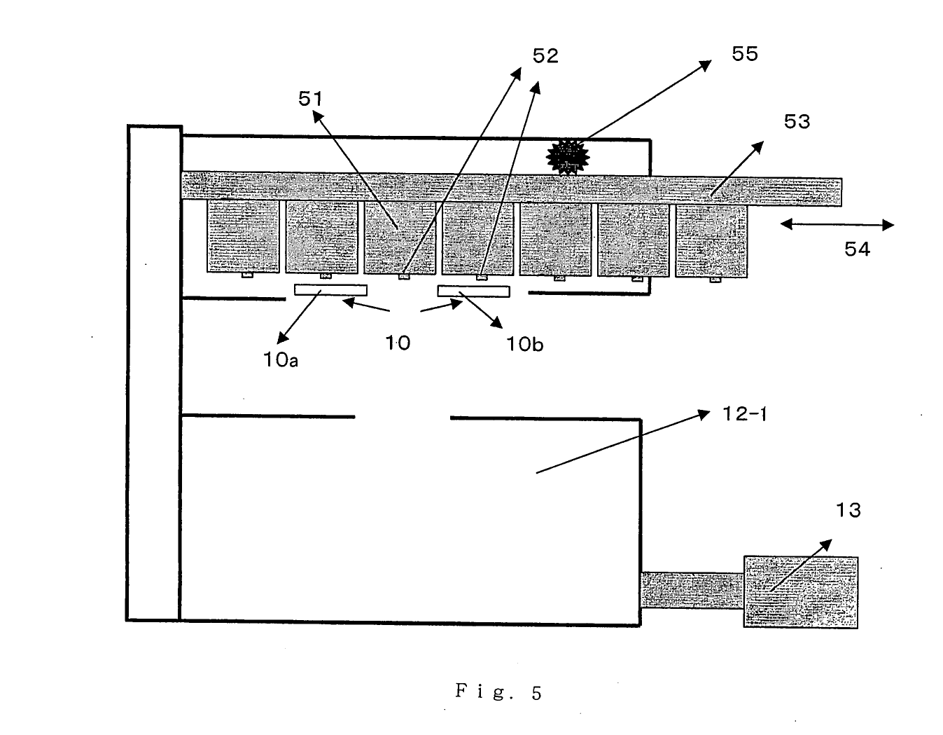

Next, the inkjet unit 53, which contains two or more tanks (an inkjet contains a tank) holding an inkjet and spotting liquid, is laid in the traverse unit B 12-1, spotting liquid of two or more kinds of probes DNA is discharged from the inkjet 51 one by one, and how to align to a pregroove on a DNA microarray disc is explained by using FIG. 5.

Here, the inkjet unit 53 on which two or more inkjets are contained as shown in FIG. 5, is constructed on the traverse unit B 12-1. The inkjet unit can be moved with the transverse unit B by the transfer gear 55. At this time two or more inkjets 51 are formed, and each inkjet is made to discharge liquid containing different ingredient of the probe DNA. Therefore each inkjet can form spots containing a different probe DNA. In order that the photodetector 10 and the inkjet nozzle 52 formed on the inkjet unit always maintains the same position relation, the inkjet unit 53 is controlled on its movement in the direction, which corresponds to the movement of the inkjet unit 54 to transverse unit B 12-1.

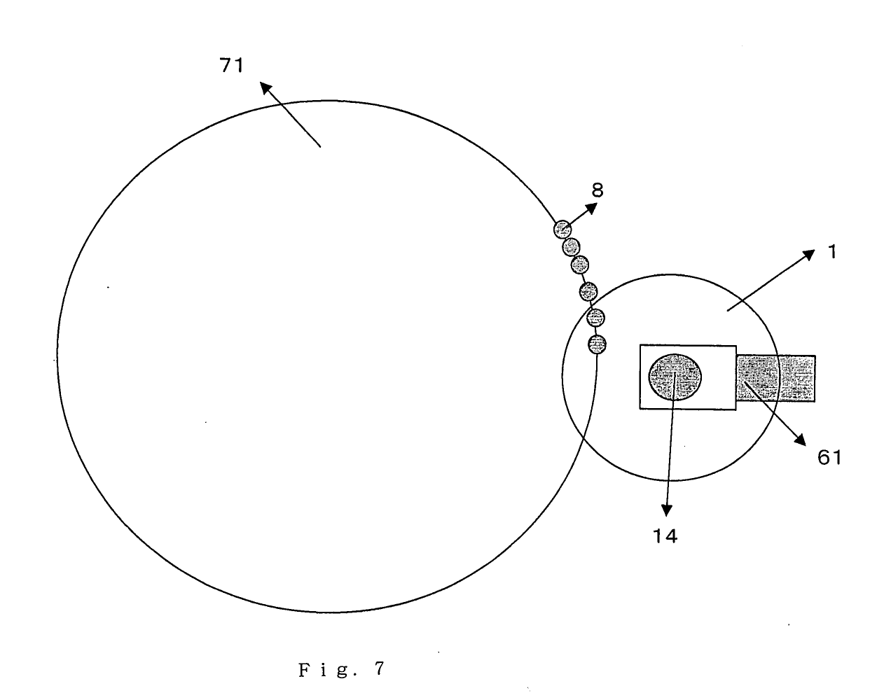

FIGS. 6 and 7 are constitutional figures showing another embodiment of the spotting apparatus. FIG. 6 is the constitutional figure from the side and FIG. 7 is the constitutional figure from the upper surface. In FIG. 6, since there are many common features with FIG. 1, same numbers are used in FIG. 6 as in FIG. 1.

An outline of the operation is described first. To discharge spotting liquid of probe DNA from the inkjet on the pregroove 23 of the DNA microarray disc 1 and to align spotting liquid on the pregroove, the DNA microarray disc is rotated. At the same time, a rotary table, on which two or more inkjets are attached, is rotated, and the position of the DNA microarray disc is moved in the transfer direction 62 so that a desired inkjet is located at the specified position on the pregroove. When the inkjet approaches to the specified position on the pregroove, spotting liquid is discharged from the inkjet and is aligned on the pregroove.

Next, a detailed operation is explained.

In FIG. 6, the elements which have a bigger number than 60 are newly added to the constitution shown in FIG. 1. The disc motor 14 is transported in the direction shown in 62 by the disc motor transfer equipment 61. The DNA microarray disc 1 is clamped by the disc motor 14, and is transported together with the disc motor 14.

The laser beam, which is outputted from the object lens 4, is diffracted by the pregroove 23, and the far-field pattern of the reflected light is received with the photodetector 7. The relative position of the pregroove 23 on the DNA microarray disc 1 and the position of the spot are detected by the differential signal of the photodetector 7a and 7b. And the traverse motor C of 13-2 and the tracking actuator 4a are controlled, so that the differential signal is set to 0. Consequently, the spot which is formed by the laser beam on the DNA microarray disc 1 follows the pregroove 23. The traverse unit C shown in 12-2 is different from the traverse unit B shown in the FIG. 5. The inkjet and the photodetector 10 are attached to the multi inkjet rotary table 71.

The pregroove 23 diffract the laser beam, which is outputted from the object lens 4, and the far-field pattern of the reflected light is received with the photodetector 10.

The differential signal of the photodetector 10a, 10b indicates the relative position of the pregroove and the spot, which the laser beam forms on the DNA microarray disc 1. And the differential signal controls the disc motor traverse unit 61 so that the position of the nozzle of inkjet 9 follows the position on the pregroove 23.

The laser beam spot which is outputted from the object lens follows the pregroove by controlling the tracking actuator 4a and traverse motor C13-2. The nozzle follows the pregroove and discharges spotting liquid on the pregroove of the DNA microarray disc.

The timing signal prepared for spotting liquid from the inkjet, is generated by the output of the V MARK detector 15 or the address information 27 which is read by the phtodetector 7. For example, a photo coupler can be used for the V MARK detector 15. Or an optical head, which irradiates light to radial direction and scans a laser beam along with a V MARK pit stream, can read the V MARK pit stream. When the photo coupler is used, the light is irradiated from one side of the substrate, and the light which penetrated the substrate is received by a photodetector at the opposite side and the V MARK is read.

When a scanning light head is used, V MARK are scanned by a light beam, and the light which penetrated the substrate is received by a photodetector at the opposite side, and V MARK is read. In addition, the scanning direction is radial direction shown in 28 of FIG. 2. The light beam can be scanned by shifting the position of the lens which irradiates the laser beam. When scanning the V MARK and detecting the position, even though the relative velocity between DNA microarray disc and laser light is near to 0, it becomes possible to read the position information of the pregroove.

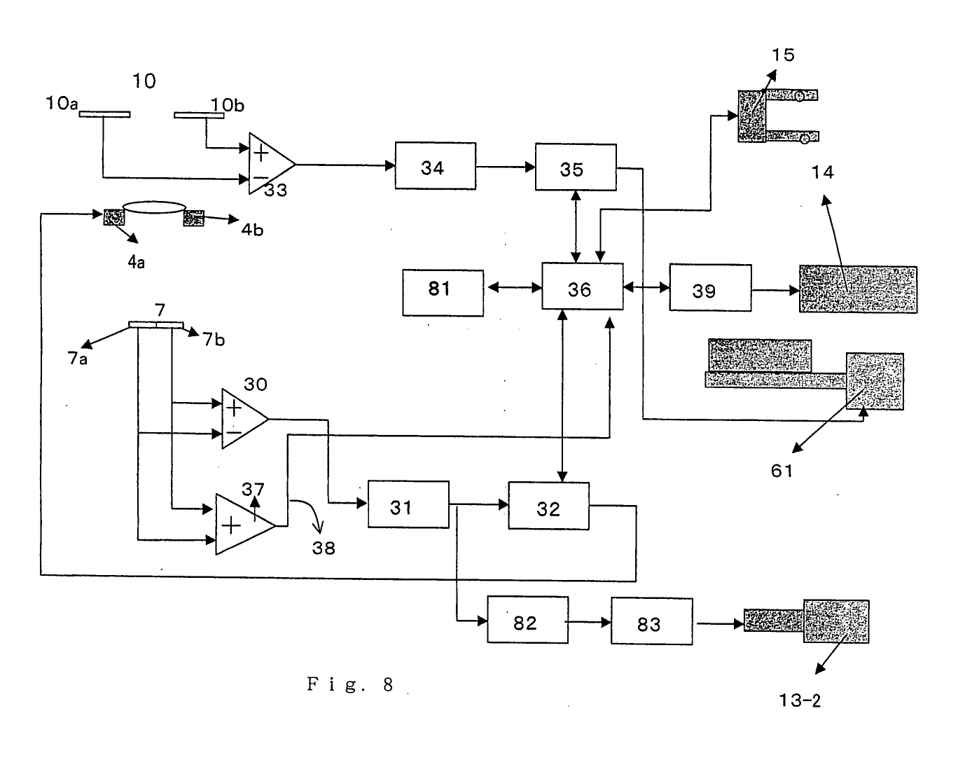

The ink jet 8 attached on the multi-inkjet-rotary table 71 in FIG. 7 discharges spotting liquid on the pregroove of DNA microarray disc 1. The rotary table rotation control equipment 83 shown in FIG. 8 controls the rotation of the multi-inkjet rotary table 71. In addition, although not illustrated, the photodetector 7 detects address information. The CPU 36 shown in FIG. 8 generates the timing signal for an inkjet discharging using the V MARK and the address information. The traverse motor C13-2 is controlled by the drive signal via the out put of the phase compensator amplifier 81 and the drive amplifier 83 which is based on the output of the phase compensator amplifier 31.

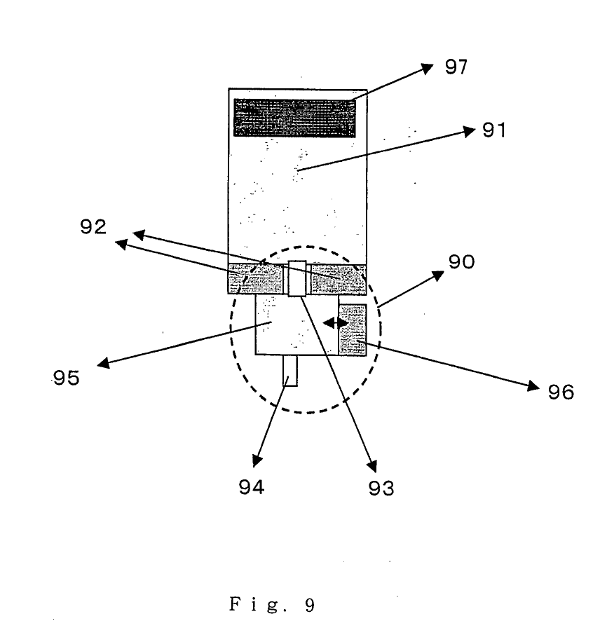

In FIG. 9, the structure of the ink jet used for the multi-spotting equipment in FIG. 7 is shown. The spotting liquid containing probe DNA is stored in the tank 91. The inkjet A, 90 is connected to the tank 91 via the hole 93.

The component 92 shows a connection seal. The pressurization device 96 gives pressure to spotting liquid at the pressurization room 95, and discharges spotting liquid from the outlet 94.

The liquid name display part 97, such as a bar code, displays the kind of spotting liquid stored in the tank 91.

Before spotting on the pregroove, the bar code 97 is read, and the name of the spotting liquid with the address information of the pregroove is recorded on a memory. The address information of the pregroove can also be read by using the adder amplifier output 38 in FIG. 3. Moreover, it is also possible to detect the spotting position on the DNA microarray disc by using the output of the V MARK detector of FIG. 6. Finally the contents of this memory are transferred to the record region on the DNA microarray disc (the region 24 of FIG. 2, or 25), and it enables to know what spotting liquid is arranged in which position on the DNA microarray disc after spotting ends.

As above mentioned, the position of pregroove on the substrate is detected and it is possible to spot the probe DNA on a pregroove in the form of spotting liquid. Moreover, the probe DNA can be provided in a position with the predetermined address information on the substrate, and the probe DNA spreading is limited by the pregroove. Therefore, it becomes possible to arrange the probe DNA with high density. In FIG. 1, spotting liquid is arranged into the region of a convex (also called “on groove”) of pregroove, it can be also arranged into the region of a concave (also called “in groove”). In this case, the spotting liquid is arranged along with the region of concave (in groove).

Moreover, since the address information 27 can be added to a pregroove beforehand, the position of the probe DNA can be specified precisely.

In the above mentioned embodiment, when spotting the probe DNA, in order to detect the position of pregroove on a substrate, from the substrate side, the laser beam with a wavelength of 780 nm is irradiated by the laser 2, and the positions of laser beam spot is measured by detecting the position of the pregroove 23 on the substrate by using the transmitted laser beam. When the thin film on substrate is prepared, the laser beam of which wavelength can penetrates substrate from the substrate side is selected and used.

After spotting probe DNAs on the substrate, cDNA to be investigated is applied to the probe DNA on the substrate. Then after cDNA hybridizes with the probe DNA, in order to detect fluorescence contained in the resulting DNA spot, the laser beam of which wavelength are about 650 nm, 530 nm, 400 nm is irradiated to the resulting spot on the substrate and reflected light is utilized to detect the fluorescence. Therefore, to increase reflected light, SiO₂ layer or gold layer is formed on the substrate. In this embodiment, gold layer is formed on the substrate and the SiO₂ layer is formed thereon. It is also possible to use Pt in place of gold, and an inorganic or organic material, which has an equivalent feature with SiO₂. Next, the function of the thin films of Au and SiO₂ formed on substrate is explained.

In FIG. 10, the horizontal axis 100 shows thickness [nm] of SiO₂ film, and the vertical axis 101 shows the ratio of the electric field intensity of Au and SiO₂ layers formed on substrate for the reference of the only polycarbonate substrate. The electric field intensity on the substrate surface in the case preparing Au and SiO₂ thin film, and the comparison value of the case preparing nothing are shown in FIG. 10. 102 shows the characteristic in the case 103 of laser light with a wavelength of 563 nm, the wavelength of 652 nm and the laser light 104 with a wavelength of 532 nm.

The thin film of SiO₂ is formed on the Au thin film in FIG. 10. When the Au thickness is set to 50 nm, the result, in which the electric field on-the-strength ratio is calculated, is shown in FIG. 10.

In case of 70 nm of SiO₂ thickness, for example, the strength ratio of a vertical axis increased 5 times. This means that the intensity of reflection light with a wavelength of 532 nm, increases 5 times compared to the case of no additional layers.

In the graph of FIG. 10, where the thickness of Au is 50 nm, the electric field intensity measured on the surface of SiO₂ becomes 5 times high in case of 70 nm of SiO₂ thickness compared to the case of no thin film. Thus, by designing that the electric field is enlarged and irradiating the laser beam on the DNA spots including the phosphor object, the quantity of the reflected light will increase and the S/N of reflected light can be improved.

It is important for the substrate surface, when spotting, to remove dirt such as a minute oil-and-fats ingredient. Therefore, by increasing the output of the laser beam, which irradiates the pregroove just before spotting, the temperature on the surface of substrate is raised and the dirt ingredient can be removed.

Although it is possible to use the laser beam for the position detection for this purpose, spotting can also be performed by preparing another laser and raising the temperature on the surface of substrate temporarily in advance of spotting.

Although the embodiments are explained using the shape of a disc as a substrate, it is also applicable to use a form, such as the shape not of a disc but a rectangle plate. Of course, pregroove can be used also in the shape not of the circumference but the form, in which straight lines are provided.

Moreover, in order to detect the position of an ink jet, a photodetector can also be attached in to the ink jet directly. It is also possible to use methods other than the position detection method using light, for example, a magnetic detection, of course. Although the case of the embodiments using the probe DNAs (single stranded DNA), such as cDNA as a spotting liquid is explained, it is applicable to any kind of forms as a spotting liquid, if it is liquefied things, such as protein.

The substrate of this invention can be also used as a substrate for producing oligo DNA by the photochemical reaction.

Next the case of producing Probe DNA made by the photochemical reaction is explained using the substrate of this invention.

As known well, DNA is the polymer of deoxyribonucleotides (also called nucleotides). This nucleotide is a compound in which deoxyribose is bonded with phosphoric acid and one of four kinds of bases, namely adenine (A), guanine (G), cytosine (C) and thymine (T). For example, forming ester bond via a phosphoric acid can combine two nucleotides, X and Y, 5′ carbon atom in deoxyribose in X and 3′ carbon atom of deoxyribose in Y.

A nucleotide has two terminal ends. One terminal end is called 5′ carbon and another end is called 3′ carbon. And the bonding between nucleotides takes place only between 5′ carbon and 3′ carbon, and take place neither between 5′ and 5′carbons nor between 3′ and 3′ carbons.

By repeating such bonding, an infinite number of nucleotides are bonded in linear state theoretically. Since one of four kinds of bases, A, G, C, and T, is contained in each nucleotide, it is possible to specify the order of these bases in DNA to form a base sequence of DNA. These are called A, G, C, and T for simplification. The DNA produced on the substrate is called probe DNA here.

The probe DNA is produced on the DNA microarray disc shown in FIG. 2. Then, a recordable zone on a DNA microarray disc is prepared, and the producing conditions and the measurement results are recorded. Furthermore, the address information which is specified by pregroove or prepits and which shows the storing region of probe DNA, and a correspondence relation with probe DNA are also recorded on the same DNA microarray disc. In the conventional application, a glass plate contained DNA micro array and the data at the time of producing are kept independently. Since it can be saved on the same DNA microarray disc, operability, reliability, and security will be improved.

Next, the producing method is explained in detail. A preferred embodiment is shown with drawings.

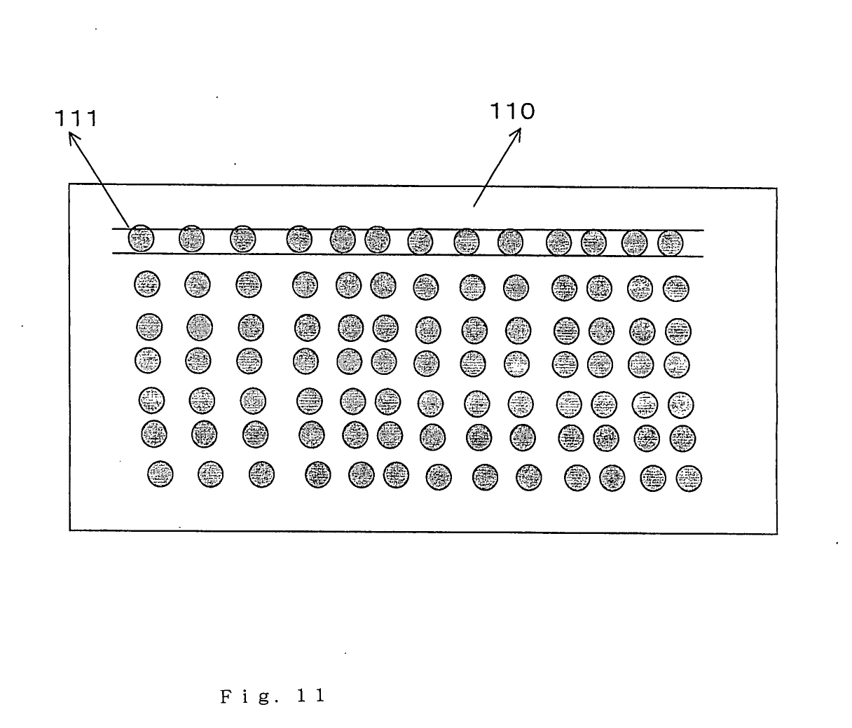

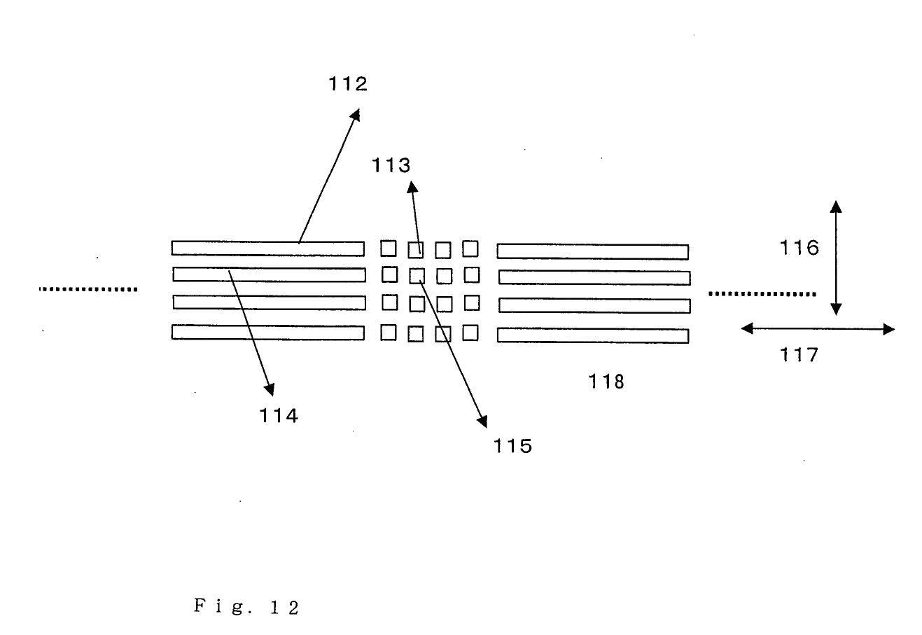

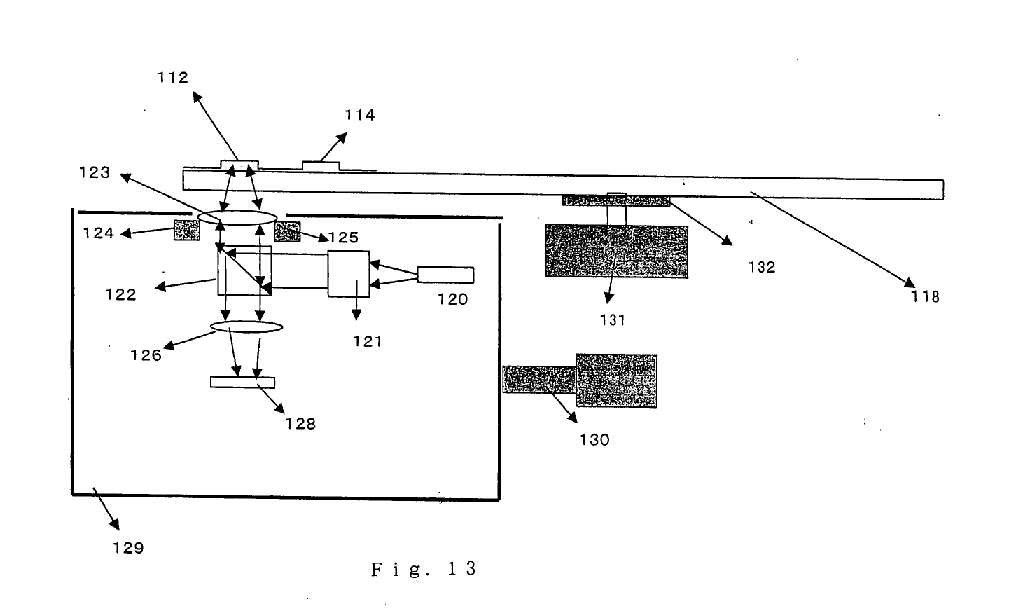

FIG. 12 and FIG. 13 illustrate the alternative embodiments of the invention. FIG. 11 is the drawing in which the region of the address which shows pregroove and its position on the DNA microarray disc is expanded and shown. And, FIG. 13 is the block diagram of the apparatus for producing the probe DNA.

An example of producing will be explained herein, using the following case. As shown in FIG. 12, the oligonucleotide is produced using four nucleotides of base arrangement called A-C-G-T on the pregroove of which address is specified by 1 shown 113. And the oligonucleotide is produced using four nucleotides of base arrangement called G-A-T-C on the pregroove of which address is specified by 2 shown 115.

As already explained, it is assumed that “A” means 3′ terminal end and “B” means 5′ terminal end. At first, the 3′ terminal end is adhered to a layer on a substrate, and nucleotides are added to the 5′-end of the nucleotide one by one. By choosing the kind of a nucleotide to be added, DNA of the base arrangement of the purpose can be produced. For example, the case where it starts from “A” is explained. Another chemical group is combined with 5′ terminal end the nucleotide, and it prevents from reacting with 3′ terminal end of other nucleotides. Such a chemistry group is called a protective group.

At first, the 3′ terminal end is covalently bound to a resin on a substrate. It is possible to use the DNA, which has biotin at the 3′-end as the probe DNA. In this case, avidin is bound to the surface of the DNA micro disc 111. And DNA is also bound to a substrate with the specific joint formation ability between avidin and biotin.

There is also the following method to bond the nucleotide of this first nucleotide “A” at the 3′ terminal end. The substance activated by photochemically is first provided on the surface of this DNA microarray disc.

Alternatively in order that the generated electric field, when laser light is irradiated on the surface of this DNA microarray disc, can be enlarged the substance of the gold layer and the layer of SiO₂, for example, can be prepared. And above on the substance, another substance, which is activated photochemically, can also be utilized.

On the surface of the DNA microdisc substrate are provided a substance activated by photochemically. And a monomer (mononucleotide) which dissociates with light irradiation and is terminated by a protective group having an OH group remained after irradiation. And the oligonucleotide is produced by bonding a monomer to fixed monomer on the substance of the substrate. Next, a laser light is irradiated on the target region of the substrate, and the protective group is removed, then the OH group is exposed on the region. After that, mononucleotide solution is applied on the region where the laser light is irradiated, then mononucleotide is bound to the OH group, consequently a polynucleotide is produced with the irradiated region. As a monomer used at this time, a 3′-O-activated phosphoryl-amidated nucleotide, whose hydroxyl group of 5′-end is photochemically protected by photosensitive protective group can be exemplified. It is explained in full detail about photochemical protected mono-nucleotides and production method therefor in WO 1997/039151 (Japanese Patent Publication (Laid open) 2000-508542). And an ortho-nitrobenzyl group may be exemplified as a photosensitive protective group.

When nucleotide solution is added to the substrate surface after irradiating a laser light, a chemical reaction will occur. And it constitutes so that it may have the activation layer to the irradiated region of which the nucleotide is bonded. For example, it is made that an OH-group is generated in the region, where is irradiated by light, and it is bound with the monomer which is applied by spin-coating apparatus.

In this invention, the storing region which can be specified by the address information constituted by a pregroove or prepits is prepared on a substrate, and a photochemical reaction is performed in the specified storing region. Therefore, the above-mentioned address information is read and laser light is irradiated selectively at the storing region.

This storing region is used as a form of a concave region or a convex region of a pregroove, a flat region, a concave pit or a convex pit with a form like a soccer stadium in a case that can be identified by prepits. Moreover, as for the size, it is desirable that width sets to 1 micrometer or more, and length sets to 1 micrometer or more at a relation with the size of laser beam spot. However, the size is not limited thereto and is set as the size, which can preserve the probe DNA. Thus, after reading address information and tracking the laser beam spot on the pregroove which is a storing region, the light is irradiated selectively at the arbitrary storing regions on the DNA microarray disc surface. Then, after irradiating laser light to the address 1, nucleotide “A” is added. The terminal end of this nucleotide “A” is protected by a protective group. A solution of nucleotide “A” is applied to a substrate by spin coating. However, the protective group used here is unstable one against a photochemical reaction. That is, when the laser light is irradiated, the deprotection reaction occurs. When this nucleotide “A” combines with the surface of pregroove 112 of the address 1, washing liquid is applied to a substrate by spin coating, and not bonded nucleotide “A” is removed.

Next after irradiating the laser light at the pregroove 114 of the address 2 to activate it photochemically, and provide the nucleotide “G”, which has a protective group at the terminal end, to react the nucleotide “A” on the said pregroove. Thus DNA microarray disc in which nucleotide “A” bond with pregroove 112 of an address 1, and nucleotide “G” bonded with pregroove 114 of an address 2 is obtained. Since the protective group of nucleotide “A” can next be removed when the laser light is irradiated at pregroove 112 of address 1, nucleotide “C” is added after that. The laser light is irradiated to pregroove 114 of the address 2 to remove the protective group of nucleotide “G”, and then nucleotide “A” is added thereto. By repeating the similar procedure after that, the DNA microarray disc which has the oligo DNA of A-C-G-T in the pregroove 112 of the address 1, and the oligo DNA of G-A-T-C in the pregroove 114 of the address 2 is produced. In addition, although the DNA microarray disc 118 is drawn linearly for convenience, it is a disc-like in practice and the pregroove is produced a concentric circle or in the shape of spiral in the tangent direction 117. And the pregroove of this concentric circle or spiral has a plurality of lines (it is several 1000 or more at track pitch 2 micrometer to about 20 micrometers) in the radial direction on the disc.

A method to irradiate a laser light to the specified pregroove is explained by using the block diagram of FIG. 13.

FIG. 13 is the block diagram of the probe DNA production equipment. 118 is the DNA microarray disc, on which the pregroove 112 of the address 1, and the pregroove 114 of the address 2 are formed. In this probe DNA production equipment, the pregroove is used as a storing region of the probe DNA.

The DNA microarray disc 118 is placed on the motor 131 through the turntable 132, and the disc motor 131 carries out the rotation control. 120 shows the laser, which is changed into parallel light by the beam expander 121, and focuses the beam spot on the pregroove 112 of the address 1 by way of the object lens 123 through the beam splitter 122. The position of the object lens 123 is controlled by the focusing actuator 125, the tracking actuator 124 and the traverse motor 130, and the laser beam, which is emitted from the object lens 123, focuses on the pregroove 112 that has the address information 1 of 113. For the servo control, the laser beam, which is outputted from the object lens, is reflected by the address 1 of 113 on the DNA microarray disc 118, and the reflected light is received by the object lens and detected with the photo-detector 128 via the lens 126. Then, using the output of the photo-detector 128, the address information is read and the position of the laser beam is controlled. When the laser beam scans to detect the pregroove of the address 1, the output power of the laser beam is held sufficient low not to affect the photo-protective group on the said pregroove. When the pregroove 112 with address 1 is detected, the output power of the laser beam is set high in order to remove the protective group. In short, during the laser beam scanning the period of the pregroove 112 with address 1, the output power of the laser is set high.

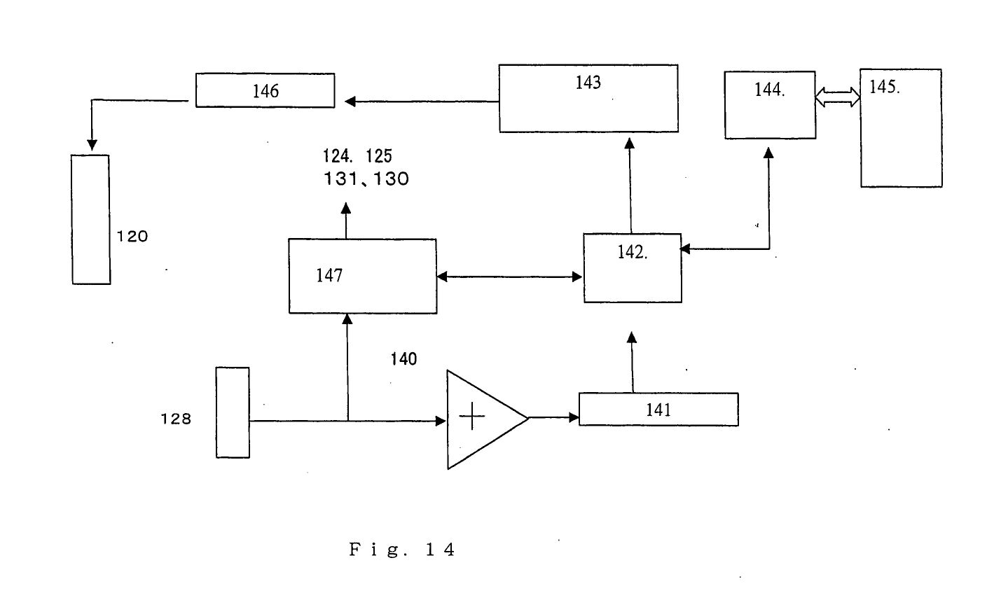

FIG. 14 is a block diagram of probes DNA production equipment. As for the laser 120, the laser power is modulated with the laser power modulator 146. The optical output from photodetector 128 is changed into electric current by the preamplifier 140, the information of the address 1 specified for the pregroove 112 is detected, it is demodulated by the decoder 141, and the address information is sent to CPU (controller) 142. After the position information of the pregroove 112 is read, the laser power which is preferable to the photochemical reaction performed on the above-mentioned pregroove is calculated by the CPU 142, and the irradiation power is outputted by controller 143. And via the laser power modulator 146, the output power of the laser 120, the output pulse width, etc. are controlled, and the laser irradiates the pregroove 112. The pulse width and the pulse peak power are controlled so that the laser power outputted from the laser 120 becomes a suitable value for the photochemical reaction. And since it is necessary to change the power in view of the relative velocity between the DNA microarray disc and the laser beam spot, the information of rotation number of the disc motor 131 is inputted into CPU 142 from the servo 147, and is used as information for the laser power control. For example, when a high laser power is required for a photochemical reaction, the relative velocity is reduced and the large intensity of optical energy (integration value of the optical energy given on pregroove) can be given on the target pregroove. The servo 147 also performs operation for the light beam from the object lens 123 to track on and focus on the pregroove of the DNA microarray disc.

In addition, PC 145 is a personal computer for controlling the probe DNA production equipment, and controls the whole operation of the probe DNA production equipment through an interface (I/F) 144. For example, the total number of the pregrooves which can be specified by using the address information on a DNA microarray disc can be made as 1 million or more places. Since it is uncontrollable to manage what kind of DNA is generated to which address only by CPU 142 inside the probe DNA production equipment, an external personal computer 145 is required.

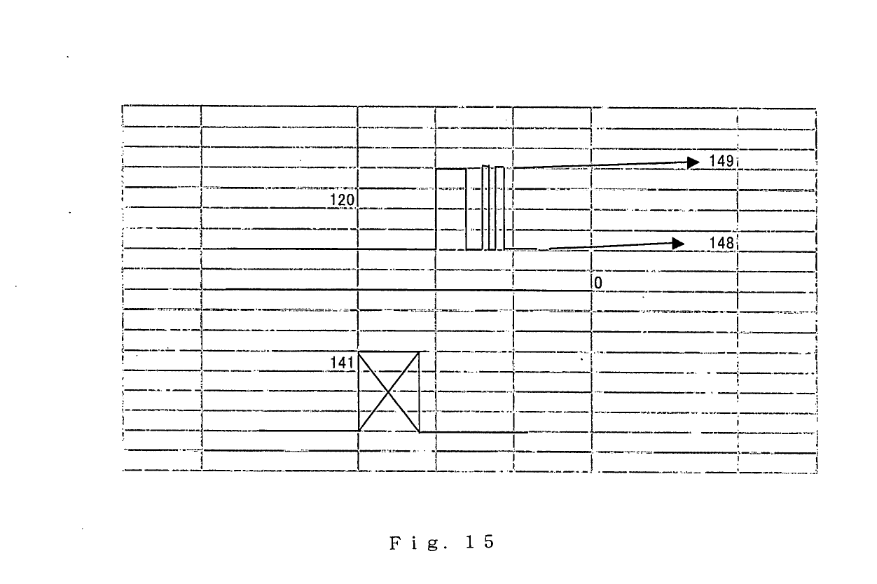

The waveform diagram is shown in FIG. 15. The 141 shows an example of the read address information. The optical output of the laser 120 reads the address information by the reproduction light power 148, and irradiates the laser light with the peak power 149 to the pregroove which has the specified address.

When exerting a photochemical reaction especially on the pregroove, the integration value of optical energy is important, and it is possible to enlarge the first pulse width of the irradiation laser power and makes the pulse width small after that, thus the suitable reaction energy can be given to the photo protective group formed on the pregroove.

Thus, the production of an “oligo DNA microarray disc” is attained by the DNA producing using the reaction of nucleotide with the protective group that is removable by de-protective reaction with irradiation of the light, and the tracking servo technology which can apply the light power to the specified small region on the DNA microarray disc surface (pregroove). It has been clarified that about 350 nm of laser wavelength is suitable to remove the protective group. Moreover, by choosing and using two or more laser wavelengths, it is also possible to change the effectiveness of photochemical reaction. For example, the reaction of a monomer can be performed selectively according to the change of the wavelength by making a photo removal protective group or a monomer to have a stronger dependency on the wavelength. By scanning a laser beam, the probe DNA finally produced can be distinguished whether the probe DNA is produced properly or not.

The method of discrimination is carried out by comparing reflectance, a color, etc. of a probe DNA with that of a proper probe DNA. And two or more probe DNAs of the same kind are produced, and thus just proper ones can be distinguished and managed. And finally, a customer can know the probe DNA which is finally qualified from the managed data, and can use the probe DNA with that address information. It is possible to keep the address information in which the normally produced probe DNA exists as a method of managing on a DNA microarray disc. Since this invention provides the DNA microarray disc with the recordable zone, it keeps information in the part.

The method analyzing mRNA using the probe DNA on the substrate finally produced as mentioned above, can be conducted according to the same process as a well-known DNA chip. After carrying out hybridization with a sample to be examined and combining the bases, the laser beam for inspection is irradiated at the probe DNA, and it carries out by observing fluorescence. Since DNA spot can be scanned to one dimension when the DNA microarray disc of this invention is used at this time, it can inspect at high speed. Since probe DNA is arranged on pregroove, high speed measurement of base bonding, improvement in operability, and low pricing are realized by being able to raise detection S/N of the probe DNA (it is called DNA spot) connected by base, and having the optical measurement part and servo part for it. And the optical measurement part, which constitutes a control part, is prepared independently to the reading optical measurement part of DNA spot, and it is able to avoid degrading the fluorescent substance contained in DNA spot by the beam spot of detecting a servo error. Moreover, as the position control of the reading beam light of a DNA microarray is performed with a sufficient accuracy using servomechanism, the error of the focus and the direction of tracking is erased, and it can realize an accurate scanning.

Moreover, in order to prevent the fluorescent substance from degradation by the reading laser beam and to enhance reading S/N of DNA spot, it is also possible to carry out amplitude modulation of the reading laser light by high frequency (for example, 100 to 500 MHz). Thus, by controlling the modulation, it turns out that accumulation of irradiation energy decreases on a substrate, and can prevent fading or degradation of the fluorescent substance.

An embodiment which shows concretely the best mode for measuring the DNA microarray disc of this invention are given below with drawings.

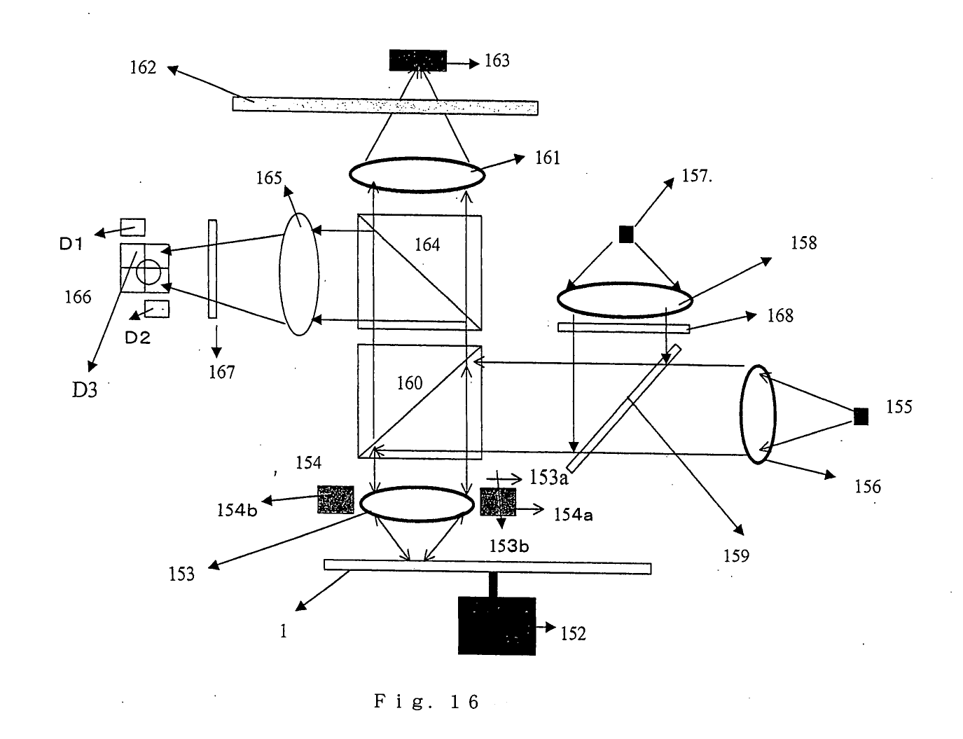

Hereafter, the outline of the examples in this invention is illustrated by using FIG. 16 and FIG. 2.

FIG. 16 is the block diagram showing the construction of the equipment which reads the DNA microarray disc shown in FIG. 2. FIG. 2 shows DNA microarray disc.

In FIG. 16, the DNA microarray disc 1 is rotated and controlled by the disc motor 152. The laser light focuses on the DNA microarray disc using the object lens 153, and irradiates the DNA spots on the DNA microarray disc, then, the reflected light is collected. The object lens 153 is controlled by the actuators composed by the focusing element 154a and the tracking element 154b, and the object lens 153 can be shifted to the direction of perpendicular to the disc (Z direction shown by 153b) and also to the radial direction to the disc (X direction shown by 153a) to follow the DNA spots sequence.

The light source, shown by 155, is used for a fluorescence excitation (output light wave length λ1), and utilizes a laser with a wavelength of 650 nm in this case. 156 is the collimator lens 1, which converts the output of the laser light 155 into a parallel light or a divergent light with a specified angle, and makes a focus on the DNA microarray disc 1 with the object lens 153 via the half mirror 159, and beam splitter 1 of 160. The reason why the output light is changed into the divergent light with the specified angle is to make the beam spot, when focused on the DNA microarray disc, to have almost a same beam spot size as the DNA spot size. It is the reason for utilizing the divergent-light, which has a specified angle. To get a adequate spot size, it is possible to put an aperture at the front of object lens for the laser beam which is outputted from a light source of wavelength λ1. As a result, the NA of the object lens decreases substantially.

The aperture can be provided in the laser beam output side of the collimator lens 156 in FIG. 14. For example, even when an object lens having the NA of 0.6 is used, the NA is 0.5 for the light having a wavelength λ1, and the NA is 0.6 for the servo light having a wavelength λ3. By the above method, it is possible to enhance a detective sensitivity of servo error, and enlarge the reading beam radius of DNA spots.

The light source for servo 157 (output wavelength λ3) focuses on the DNA microarray disc 1 via the collimator lens 2 of 158, the half mirror 159, the beam splitter 1 of 160, the object lens 153. The reflected light from the DNA microarray disc is led to the servo error photodetector 166 via the object lens 153, the beam splitter 1 of 160, the beam splitter 2 of 164 through the collecting lens 2 of 165.

On the other hand, output light (output wave length: λ1) of light source 155 for exciting fluorescence, which is focused on the DNA microarray disc 1 through the object lens 153, excites the phosphor contained in the DNA spots on the DNA microarray disc 1. The excited light having a wavelength λ2 is collected through the object lens 153. The light is collected by the lens 1 of 161 via the beam splitter 160, 164. The excited light having the wavelength λ2 only is selected by the optical filter 162, and then led to the fluorescence light detector 163. The beam splitter 1, 2 of 160, 164 are designed to pass the wave length λ2 light of the phosphor in the DNA spots which is excited by the laser having the wavelength λ1. The beam splitter 14 reflects the light having the output wavelength λ3 of the servo light source. The optical filter 162 is designed to pass the light having the wavelength λ2. The output light of the servo light source 167 is converted to the parallel light by the collimator lens 2 of 168.

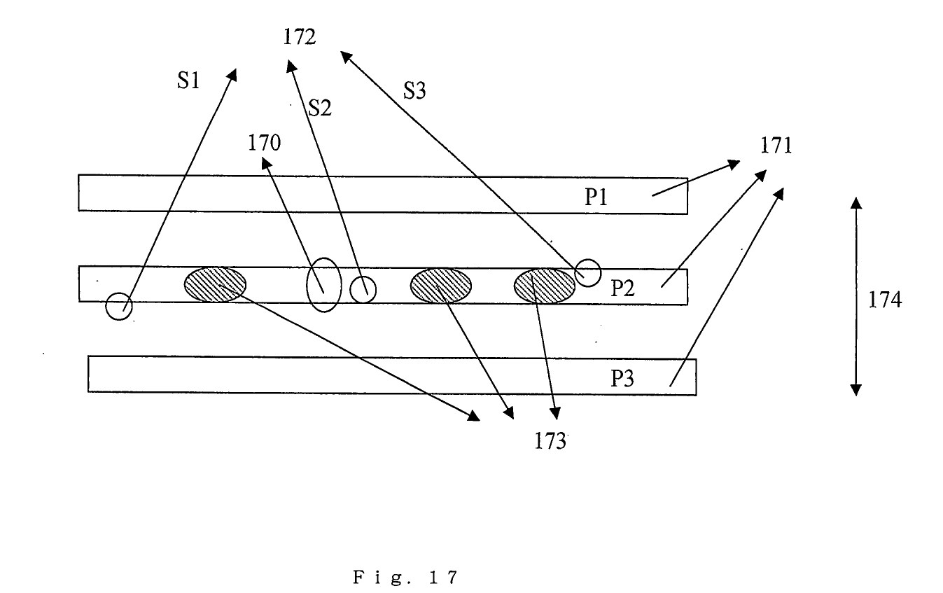

The spot changes its shape at around the focus by the cylindrical lens 167 when collected through the object lens 153. Namely, the spot changes its shape from a true circle to an ellipse on the servo error photodetector 166. This change is measured by the servo error photodetector, which includes the photodetector 166 divided into four portions. When the beam reflected from DNA microarray disc is projected at the center of the detector in a state of an approximately true circle, the object lens 153 is deemed to focus. Simultaneously, the output of the light source for the servo by way of the object lens 153 forms 3 beam spots S1, S2, S3 on the DNA spots or on the pregroove of the DNA microarray disc 1, by using the refractive optical lattice 168 shown in FIG. 17. The reflected beam by beam spots S1, S3 is received with D1, D2 in the servo error photodetector 166, and the difference of the output of D1 and D2 are utilized as a tracking error.

FIG. 17 shows a principle of mutual relationship between the DNA microarray disc and the beam spots formed by the reading laser beam and the servo laser beam.

In FIG. 17, 171 are the pregroove and are shown by P1, P2, and P3. The pregroove is set 1 to 100 μm in width, 0.1 to 10 μm in height with convex or concave, and about 1 to about 150 μm in the interval of grooves. DNA spots 173 are formed on the pregroove 171. 172 is a servo beam and is shown to irradiate the pregroove P2 of 171. The servo beam S1 irradiates the servo error photodetector D1 shown in FIG. 14, the S2 irradiates the D3, and the S3 irradiates the D2.

DNA spots reading beam 170 (wave length: λ1) is irradiated to DNA spots 173 and excites a phosphor contained in the DNA spots 173. The light having the wavelength λ2 excited by the light having the wavelength λ1 is readable by the fluorescence light detector 163 in FIG. 16. The servo beam formed from the servo light source (wave length: λ3) is irradiated to the DNA spots, but are set so that the beam does not have a wave length which excites phosphor in the DNA spots, eg. 780 nm. Thus, the beam does not affect the DNA spots.

### INDUSTRIAL APPLICABILITY

The present invention as defined above can achieve the following effects.

In case of using a DNA microarray disc in which the probe DNA is precisely spotted to a pregroove on DNA microarray disc, it is possible to increase reading speed because reading beam is sufficient to scan in a direction of one dimension. Therefore, the reading operation is completed when DNA spots are scanned one time, making the imaging by a conventional two-dimensional scanning unnecessary.

Further, many DNA spots than conventional one can be provided on the substrate, since DNA microarray disc having a pregroove is used as a substrate. For example, DNA microarray disc having more than 100,000 spots can be obtained.

Since a thin film such as gold film is provided on a substrate, and a thin film such as SiO₂ film is provided thereon, an intensity of reflection light is increased when a laser is irradiated on the substrate, giving a large S/N ratio when detecting DNA spots on a DNA microarray disc.

Further, as resin is usable as a substrate material, it is possible to lower cost for a total system. Even when resin is used as a substrate material, thin films are formed symmetrically at both surfaces of the substrate, bending of the substrate can be suppressed to a minimum at hybridization.

A spotting solution is placed in each solution tank and the tank is provided with a label display in which a kind, a name, etc. are shown, which prevents from spotting an incorrect solution.

A solution is spotted at high speed, and thus can be spotted on a DNA microarray disc by combining plural rotary tables. The spotting time per one disc can be saved and shortened.

When the probe DNA is produced by photochemical reaction on a pregroove of DNA microarray disc, a laser beam is irradiated to a pregroove having an identified address. The reaction can be conducted only by control of a laser beam, thus conventional masks are unnecessary. Further, different probe DNA can be obtained each time by control of the laser beam, giving a custom DNA chip easily.

The invention is explained with examples in which a pregroove having identified address information is prepared on a substrate. If a storing region is used in place of the pregroove, it is possible to use a storing region, which is flat, concave or convex. A storing region is prepared which can be identified by a prepit having address information, and a photochemical reaction is conducted at a specific storing region. Therefore, the laser beam is selectively irradiated to the specific storing region by reading the address information.

A storing region may be concave or convex of a pregroove, or may be flat, concave pit or convex pit such as a soccer stadium, provided that a prepit can identify it. The storing region is preferably 1 μm or more in width, 1 μm or more in length in view of relation to a current laser beam spot, but it is not limited. A storing region can be any size in so far as the probe DNA is stored.

Both of a laser beam for a servo and a laser beam for reading are used for laser beams to irradiate a DNA microarray disc or DNA microarray spot. Thus, a laser beam for reading can be precisely positioned on a DNA spot, phosphor of DNA spot can be effectively excited, and sensitivity of detection of fluorescence can be improved. Of course, even deformation such as bending of a substrate having DNA micro spots or aberration of DNA spot position is occurred, DNA position is precisely detected and is irradiated by the reading beam precisely.

Further, a phosphor is prevented from fading by modulating reading beam at high frequency. Thus, it is possible to irradiate a larger reading laser beam peak power than conventional one. Particularly, in a case of using a DNA microarray disc substrate, it is possible to increase reading speed because reading beam is sufficient to scan in a direction of one dimension. Therefore, the reading is completed when DNA spots are scanned one time, making the imaging by a conventional two-dimensional scanning unnecessary.

1-10. (canceled)

11. A method of producing a DNA microarray
which comprises forming an activated layer on a substrate which has a storing region and an address specifying the storing region on a concentric circle- or spiral-like track on a surface of the substrate, which can be tracked by an optical beam;
providing a disk motor for rotation control of the substrate;
providing an object lens which irradiates the optical beam on the track, and the optical beam follows the track by a tracking servo mechanism which controls a position of the object lens;
providing an address reading equipment which reads the address showing the storing region by the optical beam to specify the storing region;
after irradiating the activated layer in the storing region 1 using an optical power control device for controlling an irradiation power at a same or different irradiation power as reading the address 1;
adding a first nucleotide having a protective group at a terminal end at least in the storing region 1 on the substrate, bonding the first nucleotide to the first storing region specified by the address 1, and removing nucleotides other than the first nucleotide bonded in the storing region 1;
next irradiating an optical beam in the storing region 2 with the address 2 to activate the storing region 2, and bonding a second nucleotide having a protective group at a terminal end to the storing region 2;
bonding the first nucleotide to the storing region 1 with the address 1, bonding the second nucleotide to the storing region 2 with the address 2, and bonding the third nucleotide with the storing region 1 after irradiating an optical beam at the storing region 1 with the address 1 to remove a protective group of the first nucleotide, and then bonding the fourth nucleotide with the storing region 2 after irradiating an optical beam at the storing region 2 with the address 2 to remove a protective group of the second nucleotide;
repeating the same operation as above using a nucleotide having a protective group in a predetermined order and controlling similarly for the storing region with the address 3 and subsequent addresses;
and producing an objective DNA having a nucleotide sequence at the storing region specified by each address.

12. A method of producing a DNA microarray according to claim 11 wherein the activated layer is formed on the substrate by providing a substance which can be activated photochemically.

13. A method of producing a DNA microarray according to claim 11 wherein a solution containing a nucleotide is spin-coated to add the nucleotide and, a washing liquid is spin-coated to remove the nucleotide other than the nucleotide bonded.

14. A method of producing a DNA microarray which comprises forming an activated layer on a substrate which has a storing region, having and an address specifying the storing region, on a track which can be tracked by an optical beam on a surface of said substrate;
specifying the storing region by reading the address showing a predetermined region by the optical beam;
adding a first nucleotide having a protective group at the terminal end, after irradiating the activated layer in the first storing region for controlling an irradiation power at a same or different irradiation power as reading an address 1;
bonding the first nucleotide to the first storing region specified by address 1, and removing nucleotides other than the first nucleotide bonded in the storing region 1;
next irradiating an optical beam in a second storing region of the address 2 to activate the second storing region, and bonding a second nucleotide having a protective group at the terminal end to the second storing region;
bonding the first nucleotide to the first storing region of the address 1, bonding the second nucleotide to the second storing region of the address 2, and bonding a third nucleotide after irradiating an optical beam at the first storing region of the address 1 to remove the protective group of the first nucleotide, and then bonding a fourth nucleotide after irradiating an optical beam at the second storing region of the address 2 to remove the protective group of the second nucleotide;
repeating the same operation as above using a nucleotide having a protective group in a predetermined order and controlling similarly for the storing region with the address 3 and subsequent addresses; and producing an objective DNA having a nucleotide sequence at the storing region specified by each address.

15. A method of removing a protective group of nucleotide which comprises forming a storing region and an address specifying the storing region on a track which can be tracked by an optical beam on a substrate; arranging a nucleotide having a protective group in two or more storing regions; specifying the storing region by reading the address showing a predetermined region by the optical beam; irradiating the stored nucleotide having a protective group by the above-mentioned optical beam; removing the protective group of the nucleotide having the protective group stored in the storing region.

16. A method of producing a DNA microarray according to claim 11 which comprises a step of controlling a reaction of the protective group of a nucleotide by giving a wavelength dependency to the light wave length absorption characteristic of the protective group of the nucleotide and by changing the wavelength of an irradiated optical beam.

17. A method of distinguishing a DNA microarray which comprises discriminating a proper DNA by scanning the DNA of the obtained DNA microarray disk by an optical beam.

18. A method of using a DNA microarray which comprises inspecting a produced DNA, selecting an address in which a proper DNA exists, recording information of the selected address on a recordable portion of the substrate having DNA disposed thereon, and using only DNA existed in the storing region of the selected address.

19. A substrate for use in the producing method of a microarray of claim 11 characterized in that the substrate includes the address by protrudent pits and depressed pits, and has a storing region of a depressed part or a protrudent part of a pregroove or a flat region, a depressed pit or a protrudent pit with a form like a soccer stadium that can be identified by prepit.

20. A substrate for use in the producing method of a microarray of claim 11 which has a recordable portion on a specific area of the substrate.

21. A spotting apparatus for spotting liquid containing probe DNA or protein, which comprises providing a pregroove and an address information specifying thereof, on a concentric circle- or spiral-like track which can be tracked by an optical beam on a surface of a substrate, and controlling rotation of the substrate;
irradiating optical beam via an object lens at the pregroove, receiving the reflection beam with the first photodetector, after constituting tracking servo which make the position of the object lens controllable so that the irradiating beam of the object lens follow the pregroove, arranging equipment means which has nozzles discharging the spotting liquid containing probe DNA or protein at the pregroove,
forming a second photodetector which detects the beam penetrated the pregroove and which has at least two divided cells consolidatedly with discharging equipment to detect the relative position between discharging nozzle of the equipment and the pregroove,
obtaining a detection output indicating a relative position between the above-mentioned discharging nozzle and the above-mentioned pregroove using said second photodetector, controlling an optical block constituting the above tracking servo and the traverse unit motor controlling the consolidated movement of above-mentioned discharging equipment and the second photodetector by the detection output,
and arranging the spotting liquid discharged from the discharging equipment on the pregroove.

22. A substrate for use in the producing method of a microarray of claim 15, wherein a first reflecting layer is formed on the substrate, and at least one layer of light-permeable film is formed on the first reflecting layer, a refractive index of the light-permeable film being smaller than a refractive index of the substrate and larger than a refractive index of air, and the electric field intensity of reflection light is increased when an optical beam is irradiated at the substrate.

23. A substrate for use in the producing method of a microarray of claim 11, wherein a first reflecting layer is formed on the substrate, and at least one layer of light-permeable film is formed on the first reflecting layer, a refractive index of the light-permeable film being smaller than a refractive index of the substrate and larger than a refractive index of air, and the electric field intensity of reflection light is increased when optical beam is irradiated at the substrate.

24. A method of producing a DNA microarray according to claim 14 which comprises a step of controlling a reaction of the protective group of a nucleotide by giving a wavelength dependency to the light wave length absorption characteristic of the protective group of the nucleotide and by changing the wavelength of an irradiated optical beam.

25. A substrate for use in the producing method of a microarray of claim 14 characterized in that the substrate includes the address by protrudent pits and depressed pits, and has a storing region of a depressed part or a protrudent part of a pregroove or a flat region, a depressed pit or a protrudent pit with a form like a soccer stadium that can be identified by prepit.

26. A substrate for use in the producing method of a microarray of claim 15 which has a recordable portion on a specific area of the substrate.

27. A substrate for use in the producing method of a microarray of claim 14, wherein a first reflecting layer is formed on the substrate, and at least one layer of light-permeable film is formed on the first reflecting layer, a refractive index of the light-permeable film being smaller than a refractive index of the substrate and larger than a refractive index of air, and the electric field intensity of reflection light is increased when optical beam is irradiated at the substrate.
